(19) United States
(12) Patent Application Publication
(10) Pub. No.: US 2024/0331850 A1
(43) Pub. Date: Oct. 3, 2024

(54) CENTRALIZED CONTROL METHOD, METHOD FOR ASSIGNING A PATHOLOGY DATUM TO A DIGITAL PATIENT REPORT, AND RESPECTIVE CORRESPONDING SYSTEMS

(71) Applicant: DEDALUS ITALIA S.P.A., Firenze (IT)

(72) Inventors: Giorgio MORETTI, FIRENZE (IT); Roberto GIAMPIERETTI, MONZA (Monza-Brianza) (IT); Monica Moz, Primiero San Martino Di Castrozza (IT)

(21) Appl. No.: 18/126,890

(22) Filed: Mar. 27, 2023

(51) Int. Cl. G16H 40/20; G06Q 10/0639; G16H 40/40

(52) U.S. Cl. CPC G16H 40/20; G06Q 10/06393; G16H 40/40

(57) ABSTRACT

Described herein are centralized control methods comprising steps such as receiving past behavior of a plurality of functional variables, determining a forecast of future behavior, providing modified behavior of functional variables predicted to result in optimized behavior of process performance indicators, and transmitting the modified behavior of the functional variables. Also described are methods for assigning a pathology datum to a digital patient report. Systems and devices for executing the methods are also described.

### BACKGROUND OF THE DISCLOSURE

### Field of the Disclosure

The present disclosure relates to the field of systems and methods for managing the resources of one or more business units. In particular, the disclosure relates to a centralized control method, a centralized control system, a method for assigning a pathology datum to a digital patient report and a system for assigning a pathology datum to a digital patient report.

### Description of the Related Art

A management method or system should consider a plurality of information coming from a variety of sources to make good decisions. The decisions made by the management system aim to improve the overall performance of the system/entity it controls.

A non-optimal management of the resources of various business units can cause considerable inconvenience to the end user, who may therefore suffer from a low-quality service.

An example of a management system according to the prior art is shown in FIG. 1. Disadvantageously, the known management systems 100 (or methods) are not able to centrally manage the resources 102, 102′ of several business units 104, 104′. In particular, each business unit 104, 104′ is configured to manage only its resources 102, 102′ on the basis of local assessments based on its own information 106, 106′. In this way, a decision made locally by a processor 108, 108′ of a single business unit 104, 104′ could improve the operations of that business unit but could cause a deterioration of the operations of the business units as a whole.

For example, with reference to the field of care delivery, a plurality of information coming from a variety of sources should be considered to make better decisions that allows to cut waiting times, treat more patients, and/or reduce pressure on the staff.

In critical situations, it may be required to reroute patient flows, unveil unused capacity, and/or rapidly mobilize skills and resources.

A non-optimal management of the resources of various business units that are involved in the patient's medical care can cause considerable inconvenience to the patience, who may suffer from a low-quality service, such as a service involving long waiting times, high pressure on the staff, and/or lack of essential resources.

Moreover, In the prior art, there are no systems and methods that can work together with management systems/methods in order to identify the pathology afflicting a patient in the fastest and safest way.

### SUMMARY OF THE DISCLOSURE

One embodiment of a centralized control method according to the present disclosure comprises receiving the past behavior over time of a plurality of functional variables related to a specific business process; determining based on the received past behavior of the functional variables and on a predictive model a forecast of the future behavior of a set of process performance indicators; providing the received past behavior and the forecast behavior to an artificial intelligence algorithm and/or a machine learning algorithm and determining an optimized behavior of the set of process performance indicators according to a set of predefined optimization criteria; and transmitting to the business unity the modified behavior of the functional variables generated by the AI algorithm and/or the ML algorithm that cause the optimized behavior. One embodiment of a system according to the present disclosure comprises a memory configured to store computer-executable instructions, and a hardware processor in communication with the memory, wherein the computer-executable instructions, when executed by the processor, configure the processor for performing the above method.

One embodiment of a method for assigning a pathology datum to a digital patient report according to the present disclosure comprises receiving a plurality of symptom information assigned to the digital patient record, comparing the a symptom information with plurality of epidemiological symptom information, and determining the pathology datum to be assigned to the patient record, which is associated to the plurality of epidemiological symptom information having the highest degree of concordance according to a predefined statistical the received plurality of symptom information. One embodiment of a system according to the present disclosure comprises a memory configured to store computer-executable instructions, and a hardware processor in communication with the memory, wherein the computer-executable instructions, when executed by the processor, configure the processor for performing the above method.

This has outlined, rather broadly, the features and technical advantages of the present disclosure so that the detailed description that follows may be better understood. Additional features and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further features and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

Many aspects of the present disclosure can be better understood with reference to the following drawings:

FIG. 1 shows a management system according to the prior art;

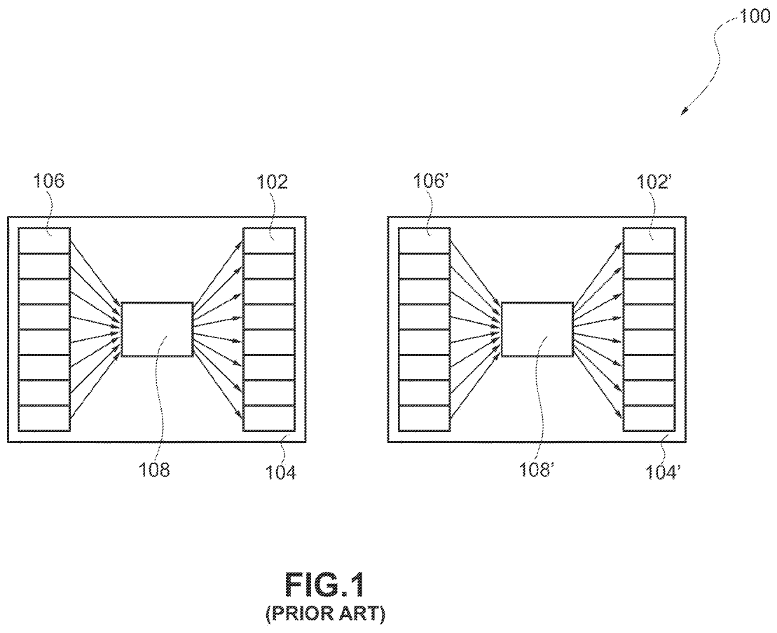

FIG. 2 shows an embodiment of a centralized control system according to the present disclosure;

FIG. 3 shows another embodiment of a centralized control system according to the present disclosure; and

FIG. 4 shows an embodiment of a system for assigning a pathology datum to a digital patient report according to the present disclosure.

The components in the drawings are not necessarily to scale, emphasis instead being placed upon clearly illustrating the principles of the present disclosure. Moreover, in the drawings, like reference numerals designate corresponding parts throughout the several views.

The drawings need to be viewed as a whole and together with the associated text in this specification. In particular, some of the drawings selectively omit features to provide greater clarity about the specific features being described. While this is done to assist the reader, it should not be taken that those features are not disclosed or are not required for the operation of the relevant embodiment.

### DETAILED DESCRIPTION OF THE DISCLOSURE

It is understood that when an element is referred to as being “on” another element, it can be directly on the other element or intervening elements may also be present. Similarly, f an element is “attached to,” “connected to,” or similar, another element, it can be directly attached/connected to the other element or intervening elements may also be present. Furthermore, relative terms such as “inner”, “outer”, “upper”, “top”, “above”, “lower”, “bottom”, “beneath”, “below”, and similar terms, may be used herein to describe a relationship of one element to another. Terms such as “higher”, “lower”, “wider”, “narrower”, and similar terms, may be used herein to describe angular and/or relative relationships. It is understood that these terms are intended to encompass different orientations of the elements or system in addition to the orientation depicted in the figures.

Although the terms first, second, etc., may be used herein to describe various elements, components, regions and/or sections, these elements, components, regions, and/or sections should not be limited by these terms. These terms are only used to distinguish one element, component, region, or section from another. Thus, unless expressly stated otherwise, a first element, component, region, or section discussed below could be termed a second element, component, region, or section without departing from the teachings of the present disclosure.

Embodiments of the disclosure are described herein with reference to view illustrations that are schematic illustrations. As such, the actual thickness of elements can be different, and variations from the shapes of the illustrations as a result, for example, of manufacturing techniques and/or tolerances are expected. Thus, the elements illustrated in the figures are schematic in nature and their shapes are not intended to illustrate the precise shape of a region and are not intended to limit the scope of the disclosure.

The following is a description of a first embodiment of a centralized control method according to the present disclosure.

In this embodiment, the centralized control method comprises the steps described in details below.
Step a): receiving, by means of at least one communication channel 202, 202′ from at least one business unit 204, 204′ of an entity, the past behavior over time of a plurality of functional variables 206, 206′ related to a specific business process to be controlled.
Step b): determining, by means of at least one hardware processor 208, based on the received past behavior of the functional variables 206, 206′ and on a predictive model related to said specific business process, a forecast of the future behavior of a set of process performance indicators of said specific business process.
Step c): providing the received past behavior of the functional variables and the forecast behavior of the set of process performance indicators to an artificial intelligence algorithm a and/or machine learning algorithm 210 configured to generate modified behavior of the functional variables adapted to cause the at least one processor to determine, based on the modified behavior of the functional variables and the predictive model related to said specific business process, an optimized behavior of the set of process performance indicators related to the specific business process to be controlled, according to a set of predefined optimization criteria;
Step d): transmitting to the at least one business unit of the at least one entity, by means of the at least one communication channel 202, 202′, the modified behavior of the functional variables generated by the artificial intelligence algorithm and/or the machine learning algorithm 210 that cause the at least one processor 208 to determine the optimized behavior of the set of process performance indicators related to the specific business process to be controlled.

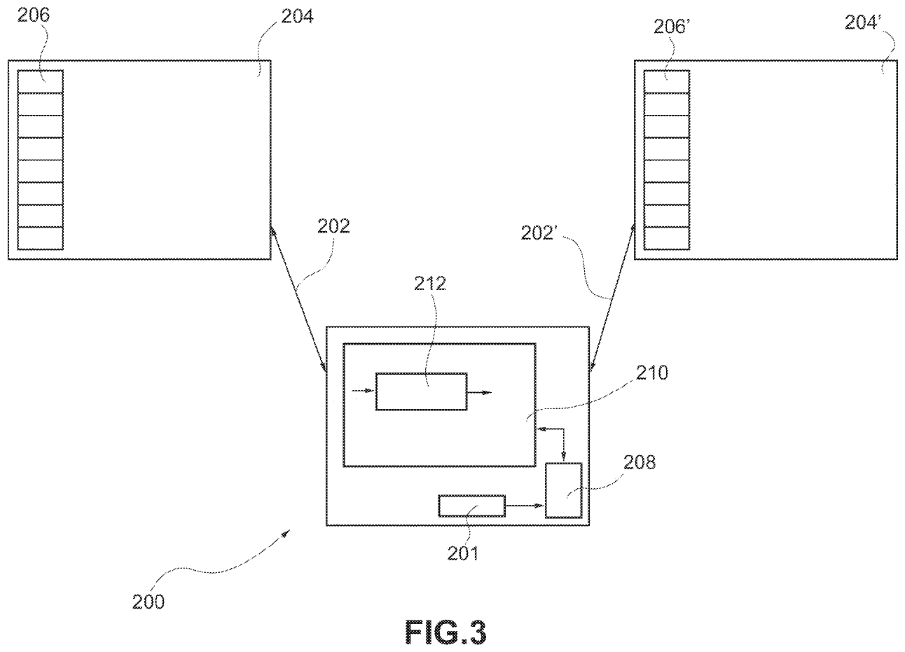

In the specific embodiment shown in FIG. 3, the artificial intelligence algorithm and/or the machine learning algorithm 210 may comprise a Kalman Filter 212. In such a case, the step c) may comprise:
c′) providing the received past behavior of the functional variables 206, 206′ and the forecast behavior of the set of process performance indicators to the Kalman Filter 212; and
c″) by means of the Kalman Filter 212, starting from the received past behavior of the functional variables 206, 206′ and the forecast behavior of the set of process performance indicators, determining the modified behavior of the functional variables adapted to cause the at least one processor to determine, based on the modified behavior of the functional variables and the predictive model related to said specific business process, the optimized behavior of the set of process performance indicators related to the specific business process to be controlled, according to a predefined set of optimization criteria.

A Kalman filter 212 is an efficient recursive filter adapted to evaluate the state of a dynamic system from a series of measurements subject to noise. Due to its intrinsic characteristics, it is a filter for noise and disturbances acting on Gaussian systems with zero mean. For example, a Kalman filter finds use as a state observer, as loop transfer recovery (LTR), and as a parametric identification system.

In some embodiments, the at least one processor may be configured to execute a Discrete-Event Simulator. In such a case, the step b) may comprise:
b′) providing the received past behavior of the functional variables over time to the Discrete-Event Simulator;
b″) by means of the Discrete-Event Simulator based on the received past behavior of the functional variables, determining a forecast of the future behavior of the set of process performance indicators of said specific business process.

In general, a discrete event simulation is a technique used in the study of system dynamics. It is a computer model where the change in the state of a system, over time, is a sequence of discrete events. Each event arrives at a given moment and modifies the state of the system.

IN some embodiments, the at least one entity may be at least one of:
a hospital;
a local health authority;
a regional health authority;
a healthcare delivery organization;
a community healthcare provider.

In some embodiments, the received functional variables 206, 206′ may comprise at least one (or in some embodiments, a plurality) of the following:
number of operators assigned to the business unit of the entity assigned to deal with the specific business process;
number of beds assigned to the business unit of the entity that are used in the provision of healthcare to patients and affect the execution of the specific business process;
number of medical devices assigned to the business 41 the entity that are used in the provision of healthcare to patients and affect the execution of the specific business process;
number of equipped rooms assigned to the business unit of the entity that are used in the provision of healthcare to patients and affect the execution of the specific business process;
amounts of consumable resources of any type assigned to the business unit of the entity that are used in the provision of healthcare to patients and affect the execution of the specific business process;
time duration requested by the business unit of the entity to execute the specific business process;
levels of cost incurred by the business unit of the entity to execute the specific business process;
levels of clinical risk incurred by the business unit of the entity to execute the specific business process;
number of critical issues incurred by the business unit of the entity that have been previously detected when the business unit of the entity has dealt with the specific business process.

In some embodiments, the set of process performance indicators of the specific business process to be controlled may comprise at least one (or in some embodiments, a plurality) of the following:
a Quality Score obtained by attributing a quality level to the execution of each task of the specific business process, considering the related medium-term clinical outcomes for the patient, such as, for example, new hospitalizations related to a specific surgical intervention;
a Time Score calculated based on the deviations of the actual process execution time from the value of a best-case execution;
a Cost Score obtained from the comparison of actual cost versus forecast cost, the latter assessed based on usage of resources as recommended by reference guidelines or best practices;
a Human Score obtained by assessing the impact of issues related to staff as detected in the actual execution of the business process to be controlled, compared to the planned execution of the same business
a Consumable, Tool, Furniture and Devices Score obtained by assessing the impact of issues related to Consumable, Tool, Furniture and Devices—for instance in terms of reduced availability or diminished efficiency—as detected in the actual execution of the business process to be controlled, compared to the planned execution of the same business process;
an Infrastructural Resources Score obtained by assessing the impact of issues related to Infrastructural Resources—for instance in terms of reduced availability or limited capacity—as detected in the actual execution of the business process to be controlled, compared to the planned execution of the same business process;
a Concise Score representing a weighted combination of the above indicators to provide an overall, single-value representation of the performance of the business process to be controlled.

In some embodiments, the specific business process may comprise at least one (or in some embodiments, a plurality) of the following tasks or sequence of tasks:
performing a surgical procedure;
performing a medical act for diagnostic purposes;
performing a medical act for therapeutic purposes;
performing a sequence of medical and non-medical acts for the purpose of assisting patients;
managing at least one space equipped for healthcare provision assigned to the business unit of the entity;
managing the personnel of the at least one business unit of the entity assigned to deal with the specific business process.

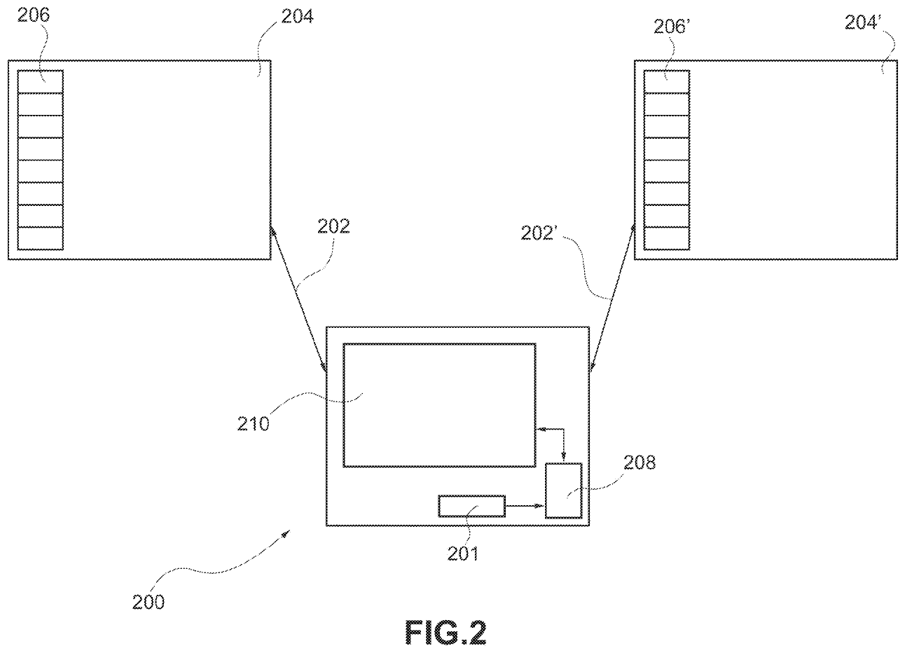

Now with reference to FIG. 2, an embodiment of a centralized control system according to the present disclosure is described.

The centralized control system 200 comprises a memory 201 configured to store computer-executable instructions. In addition, the centralized control system comprises a hardware processor 208 in communication with the memory 201.

The computer-executable instructions, when executed by the processor 208, configure the processor for:
a) receiving, by means of at least one communication channel 202, 202′ and from at least one business unit 204, 204′ of an entity, the past behavior over time of a plurality of respective functional variables 206, 206′ related to a specific business process to be controlled;
b) determining, based on the received past behavior of the functional variables 206, 206′ and on a predictive model related to said specific business process, a forecast of the future behavior of a set of process performance indicators of said specific business
c) providing the received past behavior of the functional variables 206, 206′ and the forecast behavior of the set of process performance indicators to an artificial intelligence algorithm and/or a machine learning algorithm 210 configured to generate modified behavior of the functional variables adapted to cause the at least one processor to determine, based on the modified behavior of the functional variables and the predictive model related to said specific business process, an optimized behavior of the set of process performance indicators related to the specific business process to be controlled, according to a set of predefined optimization criteria;
d) transmitting to the at least one business unit 204, 204′ of the at least one entity, by means of the at least one communication channel 202, 202′, the modified behavior of the functional variables generated by the artificial intelligence algorithm and/or a machine learning algorithm 210 that cause the at least one processor 208 to determine the optimized behavior of the set of process performance indicators related to the specific business process to be controlled.

In some embodiments, the artificial intelligence algorithm and/or the machine learning algorithm 210 may comprise a Kalman filter 212 arranged for, starting from the received behavior of the functional variables 206, 206′ and based on the forecast behavior of the set of process performance indicators, determining the modified behavior of the functional variables adapted to cause the at least one processor to determine, based on the modified behavior of the functional variables and the predictive model related to said specific business process, the optimized behavior of the set of process performance indicators related to the specific business process to be controlled. In such a case, the computer-executable instructions, when executed by the processor 208 for performing step c), may further configure the processor for:
c′) providing the received behavior of the functional variables 206, 206′ and the forecast behavior of the set of process performance indicators to the Kalman filter 212; and c″) executing the Kalman filter 212.

In some embodiments, the computer-executable instructions, when executed by the processor 208 for performing step b), may further configure the processor 208 for:
b′) providing the received past behavior of the functional variables 206, 206′ to a Discrete-Event Simulator adapted to determine a forecast of the future behavior of the set of process performance indicators of said specific business process; and b″) executing the Discrete-Event Simulator.

In some embodiments, the at least one entity associated to the business unit 204, 204′ may be at least one of:
a hospital;
a local health authority;
a regional health authority;
a healthcare delivery organization;
a community healthcare provider.

In some embodiments, the received functional variables may comprise at least one (or in some embodiments, a plurality) of the following:
number of operators assigned to the business unit of the entity assigned to deal with the specific business process;
number of beds assigned to the business unit of the entity that are used in the provision of healthcare to patients and affect the execution of the specific business process;
number of medical devices assigned to the business unit of the entity that are used in the provision of healthcare to patients and affect the execution of the specific business process;
number of equipped rooms assigned to the business unit of the entity that are used in the provision of healthcare to patients and affect the execution of the specific business process;
amounts of consumable resources of any type assigned to the business unit of the entity that are used in the provision of healthcare to patients and affect the execution of the specific business process;
time duration requested by the business unit of the entity to execute the specific business process;
levels of cost incurred by the business unit of the entity to execute the specific business process;
levels of clinical risk incurred by the business unit of the entity to execute the specific business process;
number of critical issues incurred by the business unit of the entity that have been previously detected when the business unit of the entity has dealt with the specific business process.

Preferably, the set of process performance indicators of the specific business process to be controlled may comprise at least one (or in some embodiments, a plurality) of the following:
a Quality Score obtained by attributing a quality level to the execution of each task of the specific business process, considering the related medium-term clinical outcomes for the patient, such as, for example, new hospitalizations related to a specific surgical intervention;
a Time Score calculated based on the deviations of the actual process execution time from the value of a best-case execution;
a Cost Score obtained from the comparison of actual cost versus forecast cost, the latter assessed based on usage of resources as recommended by reference guidelines or best practices;
a Human Score obtained by assessing the impact of issues related to staff as detected in the actual execution of the business process to be controlled, compared to the planned execution of the same business process;
a Consumable, Tool, Furniture and Devices Score obtained by assessing the impact of issues related to Consumable, Tool, Furniture and Devices—for instance in terms of reduced availability or diminished efficiency—as detected in the actual execution of the business process to be controlled, compared to the planned execution of the same business process;
an Infrastructural Resources Score obtained by assessing the impact of issues related to Infrastructural Resources—for instance in terms of reduced availability or limited capacity—as detected in the actual execution of the business process to be controlled, compared to the planned execution of the same business process;
a Concise Score representing a weighted combination of the above indicators to provide an overall, single-value representation of the performance of the business process to be controlled.

Preferably, the specific business process may comprise at least one (or in some embodiments, a plurality) of the following tasks or sequence of tasks:
performing a surgical procedure;
performing a medical act for diagnostic purposes;
performing a medical act for therapeutic purposes;
performing a sequence of medical and non-medical acts for the purpose of assisting patients;
managing at least one space for equipped healthcare provision assigned to the at least one business unit at the at least one entity;
managing the personnel of the at least business unit at the at least one entity assigned to deal with the specific task.

For example, a business unit may be or may comprise an electronic unit/electronic means of department that develops and implements strategies in an entity.

For example, a communication channel may be a wired communication channel or a wireless communication channel. A communication channel, e.g. in the context of telecommunications, may be a communication or propagation pathway of at least one signal or datum.

In a further aspect, the present disclosure relates to a method for assigning a pathology datum to a digital patient report.

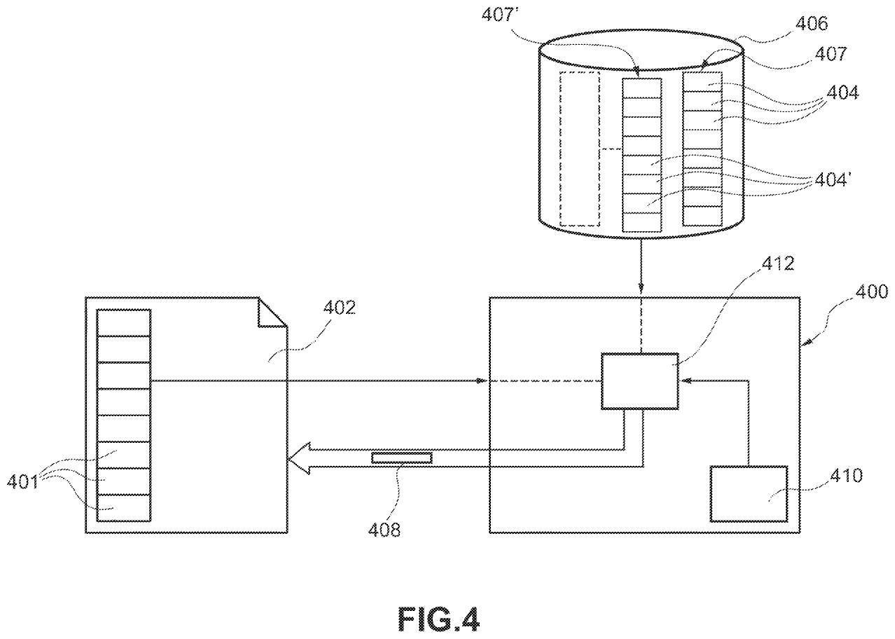

A first embodiment of a method for assigning a pathology datum to a digital patient report is described below with reference to FIG. 4.

In this embodiment, the method for assigning a pathology datum to a digital patient report comprises the steps described in details below.
Step a): receiving a plurality of symptom information 401 assigned to the digital patient record 402;
Step b): comparing the received plurality of symptom 401 information with a plurality of epidemiological symptom information 404, 404′ associated to respective pathology data 407, 407′ that are stored in a database 406;
Step c): determining that the pathology datum 408 to be assigned to the patient record is the pathology datum stored in the database 406 that is associated to the plurality of epidemiological symptom information having the highest degree of concordance according to a predefined statistical criterion with the received plurality of symptom information assigned to the digital patient record.

Preferably, a weight value may be assigned to each epidemiological symptom information. In such a case, step c) may comprise:
for each pathology datum stored in the database, determining a respective degree of concordance between the plurality of epidemiological symptom information associated to the respective pathology datum and the received plurality of symptom information assigned to the digital patient record based on the weight value assigned to each epidemiological symptom information; and
determining that the pathology datum associated to the plurality of historical symptom information having the highest degree of concordance with the received plurality of symptom information assigned to the digital patient record is the pathology datum for which the highest respective degree of concordance has been determined.

In some embodiments, the method for assigning a pathology datum to a digital patient report may also comprise the following step:
Step d): once the pathology datum to be assigned to the digital patient record has been determined, updating the epidemiological symptom information associated to such pathology datum stored in the database according to the received plurality of symptom information assigned to the digital patient record.

With reference to FIG. 4, an embodiment of a system for assigning a pathology datum to a digital patient report 400 according to the present disclosure is described below.

The system comprises a memory 410 configured to store computer-executable instructions. In addition, the system comprises a hardware processor 412 in communication with the memory. The computer-executable instructions, when executed by the processor, configure the processor 412 for:
receiving a plurality of symptom information 401 assigned to a digital patient record 402;
comparing the received plurality of symptom information 401 with a plurality of epidemiological symptom information 404, 404′ associated to respective pathology data 407, 407′ that are stored in a database 406;
determining that the pathology datum 407, 407′ to be assigned to the patient record 402 is the pathology datum 407, 407′ stored in the database 406 that is associated to the plurality of epidemiological symptom information 404, 404′ having the highest degree of concordance according to predefined statistical criterion with the received plurality of symptom information 401 assigned to the digital patient record 402.

In some embodiments, the computer-executable instructions, when executed by the processor 412, may further configure the processor 412 for:
assigning a weight value to each statistical symptom information;
for each pathology datum 407, 407′ stored in the database, determining a respective degree of concordance between the plurality of statistical symptom information 404, 404′ associated to the respective pathology datum and the received plurality of symptom information 401 assigned to the digital patient record 402 based on the weight value assigned to each statistical symptom information;
determining that the pathology datum associated to the plurality of epidemiological symptom information having the highest degree of concordance with the received plurality of symptom information assigned to the digital patient record is the pathology datum for which the highest respective degree of concordance has been determined.

Reference throughout this description to features, advantages, or similar terms does not imply that all the features and advantages that may be realized should be in any single embodiment.

Various aspects and embodiments of a centralized control method, a method for assigning a pathology datum to a digital patient report and respective corresponding systems according to the disclosure have been described. It is understood that each embodiment may be combined with any other embodiment. Furthermore, the disclosure is not limited to the described embodiment, but may be varied within the scope defined by the appended claims.

It is understood that embodiments presented herein are meant to be exemplary. Embodiments of the present disclosure can comprise any combination of compatible features shown in the various figures, and these embodiments should not be limited to those expressly illustrated and discussed. For instance and not by way of limitation, the appended claims could be modified to be multiple dependent claims so as to combine any combinable combination of elements and/or steps within a claim set, or from differing claim sets.

Although the present disclosure has been described in detail with reference to certain preferred configurations thereof, other versions are possible. Therefore, the spirit and scope of the disclosure should not be limited to the versions described above.

The foregoing is intended to cover all modifications and alternative constructions falling within the spirit and scope of the disclosure as expressed in the appended claims, wherein no portion of the disclosure is intended, expressly or implicitly, to be dedicated to the public domain if not set forth in the claims.

We claim:

1. A centralized control method comprising the steps of:
a) receiving, by means of at least one communication channel, from at least one business unit of an entity, the past behavior over time of a plurality of functional variables related to a specific business process to be controlled;
b) determining, by means of at least one hardware processor, based on the received past behavior of the functional variables and on a predictive model related to said specific business process, a forecast of the future behavior of a set of process performance indicators of said specific business process;
c) providing the received past behavior of the functional variables and the forecast behavior of the set of process performance indicators to an artificial intelligence algorithm and/or a machine learning algorithm configured to generate modified behavior of the functional variables adapted to cause the at least one processor to determine, based on the modified behavior of the functional variables and the predictive model related to said specific business process, an optimized behavior of the set of process performance indicators related to the specific business process to be controlled, according to a set of predefined optimization criteria; and
d) transmitting to the at least one business unit of the at least one entity, by means of the at least one communication channel, the modified behavior of the functional variables generated by the artificial intelligence algorithm and/or the machine learning algorithm that cause the at least one processor to determine the optimized behavior of the set of process performance indicators related to the specific business process.

2. The centralized control method according to claim 1, wherein the artificial intelligence algorithm and/or the machine learning algorithm comprises a Kalman Filter; and
wherein step c) comprises:
c′) providing the received past behavior of the functional variables and the forecast behavior of the set of process performance indicators to the Kalman Filter;
c″) by means of the Kalman Filter, starting from the received past behavior of the functional variables and the forecast behavior of the set of process performance indicators, determining the modified behavior of the functional variables adapted to cause the at least one processor to determine, based on the modified behavior of the functional variables and the predictive model related to said specific business process, the optimized behavior of the set of process performance indicators related to the specific business process, according to a predefined set of optimization criteria.

3. The centralized control method according to claim 1, wherein the at least one processor is configured to execute a Discrete-Event Simulator; and
wherein step b) comprises:
b′) providing the received past behavior of the functional variables over time to the Discrete-Event Simulator;
b″) by means of the Discrete-Event Simulator based on the received past behavior of the functional variables, determining a forecast of the future behavior of the set of process performance indicators of said specific business process.

4. The centralized control method according to claim 1, wherein the at least one entity is at least one of:
a hospital;
a local health authority;
a regional health authority;
a healthcare delivery organization;
a community healthcare provider.

5. The centralized control method according to claim 1, wherein the received functional variables comprise at least one of the following:
number of operators assigned to the business unit of the entity assigned to deal with the specific business process;
number of beds assigned to the business unit of the entity that are used in the provision of healthcare to patients and affect the execution of the specific business process;
number of medical devices assigned to the business unit of the entity that are used in the provision of healthcare to patients and affect the execution of the specific business process;
number of equipped rooms assigned to the business unit of the entity that are used in the provision of healthcare to patients and affect the execution of the specific business process;
amounts of consumable resources of any type assigned to the business unit of the entity that are used in the provision of healthcare to patients and affect the execution of the specific business process;
time duration requested by the business unit of the entity to execute the specific business process;
levels of cost incurred by the business unit of the entity to execute the specific business process;
levels of clinical risk incurred by the business unit of the entity to execute the specific business process;
number of critical issues incurred by the business unit of the entity that have been previously detected when the business unit of the entity has dealt with the specific business process.

6. The centralized control method according to claim 1, wherein the set of process performance indicators of the specific business process to be controlled comprises at least one of the following:
a Quality Score obtained by attributing a quality level to the execution of each task of the specific business process, taking into account the related medium-term clinical outcomes for the patient;
a Time Score calculated based on the deviations of the actual process execution time from the value of a best-case execution;
a Cost Score obtained from the comparison of actual cost versus forecast cost, the latter assessed based on usage of resources as recommended by reference guidelines or best practices;
a Human Score obtained by assessing the impact of issues related to staff as detected in the actual execution of the business process to be controlled, compared to the planned execution of the same business process;
a Consumable, Tool, Furniture and Devices Score obtained by assessing the impact of issues related to Consumable, Tool, Furniture and Devices—for instance in terms of reduced availability or diminished efficiency—as detected the actual execution of the business process to be controlled, compared to the planned execution of the same business process;
an Infrastructural Resources Score obtained by assessing the impact of issues related to Infrastructural Resources—for instance in terms of reduced availability or limited capacity—as detected in the actual execution of the business process to be controlled, compared to the planned execution of the same business process;
a Concise Score representing a weighted combination of the above indicators to provide an overall, single-value representation of the performance of the business process to be controlled.

7. The centralized control method according to claim 1, wherein the specific business process comprises at least one of the following tasks or sequence of tasks:
performing a surgical procedure;
performing a medical act for diagnostic purposes;
performing a medical act for therapeutic purposes;
performing a sequence of medical and non-medical acts for the purpose of assisting patients;
managing at least one space equipped for healthcare provision assigned to the business unit of the entity;
managing the personnel of the at least one business unit of the entity assigned to deal with the specific business process.

8. A system, comprising:
a memory configured to store computer-executable instructions; and
a hardware processor in communication with the memory, wherein the computer-executable instructions, when executed by the processor, configure the processor for:
a) receiving, by means of at least one communication channel and from at least one business unit of an entity, the past behavior over time of a plurality of respective functional variables related to a specific business process to be controlled;
b) determining, based on the received past behavior of the functional variables and on a predictive model related to said specific business process, a forecast of the future behavior of a set of process performance indicators of said specific business process;
c) providing the received past behavior of the functional variables and the forecast behavior of the set of process performance indicators to an artificial intelligence algorithm and/or a machine learning algorithm configured to generate modified behavior of the functional variables adapted to cause the at least one processor to determine, based on the modified behavior of the functional variables and the predictive model related to said specific business process, an optimized behavior of the set of process performance indicators related to the specific business process, according to a set of predefined optimization criteria; and
d) transmitting to the at least one business unit of the at least one entity, by means of the at least one communication channel, the modified behavior of the functional variables generated by the artificial intelligence algorithm and/or a machine learning algorithm that cause the at least one processor to determine the optimized behavior of the set of process performance indicators related to the specific business process.

9. The system according to claim 8, wherein the artificial intelligence algorithm and/or the machine learning algorithm comprise a Kalman filter arranged to, starting from the received behavior of the functional variables and based on the forecast behavior of the set of process performance indicators, determine the modified behavior of the functional variables adapted to cause the at least one processor to determine, based on the modified behavior of the functional variables and the predictive model related to said specific business process, the optimized behavior of the set of process performance indicators related to the specific business process;
wherein the computer-executable instructions, when executed by the processor for performing step c), further configure the processor for:
c′) providing the received behavior of the functional variables and the forecast behavior of the set of process performance indicators to the Kalman filter; and
c″) executing the Kalman filter.

10. The system according to claim 8, wherein the computer-executable instructions, when executed by the processor for performing step b), further configure the processor for:
b′) providing the received past behavior of the functional variables to a Discrete-Event Simulator adapted to determine a forecast of the future behavior of the set of process performance indicators of said specific business process; and
b″) executing the Discrete-Event Simulator.

11. The system according to claim 8, wherein the at least one entity associated to the business unit is at least one of:
a hospital;
a local health authority;
a regional health authority;
a healthcare delivery organization;
a community healthcare provider.

12. The system according to claim 8, wherein the received functional variables comprise at least one of the following:
number of operators assigned to the business unit of the entity assigned to deal with the specific business process;
number of beds assigned to the business unit of the entity that are used in the provision of healthcare to patients and affect the execution of the specific business process;
number of medical devices assigned to the business unit of the entity that are used in the provision of healthcare to patients and affect the execution of the specific business process;
number of equipped rooms assigned to the business unit of the entity that are used in the provision of healthcare to patients and affect the execution of the specific business process;
amounts of consumable resources of any type assigned to the business unit of the entity that are used in the provision of healthcare to patients and affect the execution of the specific business process;
time duration requested by the business unit of the entity to execute the specific business process;
levels of cost incurred by the business unit of the entity to execute the specific business process;
levels of clinical risk incurred by the business unit of the entity to execute the specific business process;
number of critical issues incurred by the business unit of the entity that have been previously detected when the business unit of the entity has dealt with the specific business process.

13. The system according to claim 8, wherein the set of process performance indicators of the specific business process to be controlled comprises at least one of the following:
a Quality Score obtained by attributing a quality level to the execution of each task of the specific business process, taking into account the related medium-term clinical outcomes for the patient;
a Time Score calculated based on the deviations of the actual process execution time from the value of a best-case execution;
a Cost Score obtained from the comparison of actual cost versus forecast cost, the latter assessed based on usage of resources as recommended by reference guidelines or best practices;
a Human Score obtained by assessing the impact of issues related to staff as detected in the actual execution of the business process to be controlled, compared to the planned execution of the same business process;
a Consumable, Tool, Furniture and Devices Score obtained by assessing the impact of issues related to Consumable, Tool, Furniture and Devices—for instance in terms of reduced availability or diminished efficiency—as detected in the actual execution of the business process to be controlled, compared to the planned execution of the same business process;
an Infrastructural Resources Score obtained by assessing the impact of issues related to Infrastructural Resources—for instance in terms of reduced availability or limited capacity—as detected in the actual execution of the business process to be controlled, compared to the planned execution of the same business process;
a Concise Score representing a weighted combination the above indicators to provide an overall, single-value representation of the performance of the business process to be controlled.

14. The system according to claim 8, wherein the specific business process comprises at least one of the following tasks or sequence of tasks:
performing a surgical procedure;
performing a medical act for diagnostic purposes;
performing a medical act for therapeutic purposes;
performing a sequence of medical and non-medical acts for the purpose of assisting patients;
managing at least one space equipped for healthcare provision assigned to the at least one business unit at the at least one entity;
managing the personnel of at the least business unit at the at least one entity assigned to deal with the specific task.

15. A method for assigning a pathology datum to a digital patient report, comprising:
a) receiving a plurality of symptom information assigned to the digital patient record;
b) comparing the received plurality of symptom information with a plurality of epidemiological symptom information associated to respective pathology data that are stored in a database; and
c) determining that the pathology datum to be assigned to the patient record is the pathology datum stored in the database that is associated to the plurality of epidemiological symptom information having the highest degree of concordance according to a predefined statistical criterion with received plurality of symptom information assigned to the digital patient record.

16. The method for assigning a pathology datum to a digital patient report according to claim 15, wherein a weight value is assigned to each epidemiological symptom information;
wherein step c) comprises:
for each pathology datum stored in the database, determining a respective degree of concordance between the plurality of epidemiological symptom information associated to the respective pathology datum and the received plurality of symptom information assigned to the digital patient record based on the weight value assigned to each epidemiological symptom information; and
determining that the pathology datum associated to the plurality of historical symptom information having the highest degree of concordance with the received plurality of symptom information assigned to the digital patient record is the pathology datum for which the highest respective degree of concordance has been determined.

17. The method for assigning a pathology datum to a digital patient report according to claim 15, comprising the step:
d) once the pathology datum to be assigned to the digital patient record has been determined, updating the epidemiological symptom information associated to such pathology datum stored in the database according to the received plurality of symptom information assigned to the digital patient record.

18. A system, comprising:
a memory configured to store computer-executable instructions; and
a hardware processor in communication with the memory, wherein the computer-executable instructions, when executed by the processor, configure the processor for:
receiving a plurality of symptom information assigned to a digital patient record;
comparing the received plurality of symptom information with a plurality of epidemiological symptom information associated to respective pathology data that are stored in a database; and
determining that the pathology datum to be assigned to the patient record is the pathology datum stored in the database that is associated to the plurality of epidemiological symptom information having the highest degree of concordance according to predefined statistical criterion with the received plurality of symptom information assigned to the digital patient record.

19. The system according to claim 18, wherein the computer-executable instructions, when executed by the processor, further configure the processor for:
assigning a weight value to each statistical symptom information;
for each pathology datum stored in the database, determining a respective degree of concordance between the plurality of statistical symptom information associated to the respective pathology datum and the received plurality of symptom information assigned to the digital patient record based on the weight value assigned to each statistical symptom information; and
determining that the pathology datum associated to the plurality of epidemiological symptom information having the highest degree of concordance with the received plurality of symptom information assigned to the digital patient record is the pathology datum for which the highest respective degree of concordance has been determined.
